(19) United States
(12) Patent Application Publication
(10) Pub. No.: US 2011/0112165 A1
(43) Pub. Date: May 12, 2011

(54) ATORVASTATIN-ALISKIREN

(76) Inventors: Andrej Kocijan, Ljubljana (SI); Matjaz Kracun, Ljubljana (SI); Andrej Bastarda, Vrhnika (SI); Rok Grahek, Kranj (SI); Darko Kocjan, Ljubljana (SI)

(21) Appl. No.: 12/863,104

(22) PCT Filed: Jan. 13, 2009
(86) PCT No.: PCT/EP2009/050299

§ 371 Date: Jan. 17, 2011

(30) Foreign Application Priority Data
Jan. 15, 2008 (EP) 08100468.1

(51) Int. Cl. A61K 31/40; C07D 207/34; A61P 9/00; A61P 3/06; A61P 9/10; G01N 30/02

(52) U.S. Cl. 514/423; 548/537; 73/61.55

(57) ABSTRACT

A new compound in which atorvastatin and aliskiren are covalently bound, salts of this compound, pharmaceutical compositions thereof, as well the use of this compound as a medicament and as an impurity standard are provided. Furthermore, a 0 process for the preparation of a compound in which atorvastatin and aliskiren are covalently bound, a method for analyzing a sample of atorvastatin and/or aliskiren, and a method for determining the retention time on a chromatographic system for atorvastatin and/or aliskiren are provided.

The present invention relates to a new compound in which atorvastatin and aliskiren are covalently bound, salts of this compound, pharmaceutical compositions thereof, as well the use of this compound as a medicament and its use as an impurity standard. Furthermore, the invention relates to a process for the preparation of a compound in which atorvastatin and aliskiren are covalently bound, and a method for analyzing a sample of atorvastatin and/or aliskiren.

All drugs contain impurities. Organic impurities can origin from raw materials, reagents and intermediates used in synthesis or bio-synthesis of drug or they can be synthetic or biosynthetic by-products or degradation products. It is important to know that impurities can affect safety and also efficacy of the drug. Therefore a high level of purity of drug substance and knowing the impurity profile are important criterions in manufacturing of a safe and efficient drug. In general, drug impurities in excess of 0.1% should be identified and quantified by selective methods. The present invention provides a atorvastain-aliskiren by-product which can be used as an impurity standard for analysis of a new combined finished dosage form (FDF) of atorvastain-aliskiren and give the opportunity to complete the characterization of impurity (response factor, toxicological studies).

The new compound may be also used as a mutual prodrug of atorvastatin and aliskiren.

HMG-CoA reductase inhibitors are known to be pharmaceutically active substances which are sensitive to the pH of the environment, humidity, light, temperature, carbon dioxide and oxygen. They are known as effective therapeutically active substances for the treatment of dyslipidemias and cardiovascular diseases, selected from the group consisting of dyslipidemia, hyperlipidemia, hypercholesterolemia, atherosclerosis (or arteriosclerosis), coronary artery diseases, coronary heart disease and the like, associated with the metabolism of lipids and cholesterol.

The mechanism of action of statin compounds is by the inhibition of the biosynthesis of cholesterol and other sterols in the liver of humans or animals. They are competitive inhibitors of HMG-CoA reductase or 3-hydroxy-3-methyl-glutaryl-coenzyme A reductase, an enzyme which catalyses the conversion of HMG-CoA to mevalonate in the liver of humans or animals, which is an important step in the biosynthesis of cholesterol in the liver. Recent studies indicate that, in addition to the above-mentioned therapeutic effects, statins also have other therapeutic effects and, accordingly, they are useful in the treatment of diseases, abnormal conditions and disorders which are selected from the group consisting of vascular disorders, inflammatory disease, allergic disease, neurodegenerative disease, malignant disease, viral disease (WO 0158443), abnormal bone states, (WO 0137876), amyloid-[beta] precursor protein processing disorders such as Alzheimer's disease or Down's Syndrome (WO 0132161).

Atorvastatin, which has the chemical name (R—(R*,R*))-2-(4-fluorophenyl)-[beta],[delta]-dihydroxy-5-(1-methylethyl)-3-phenyl-4((phenylamino)carbonyl)-1H-pyrol-1-heptanoic acid, is known as an inhibitor of 3-hydroxy-3-methylglutaryl-coenzyme A (HMG-CoA) reductase. The free carboxylic acid form of atorvastatin exists predominantly as the lactone and is disclosed in European Patent No. 0 247 633.

Aliskiren, which has the chemical name (2S,4S,5S,7S)-5-Amino-N-(2-carbamoyl-2-methylpropyl)-4-hydroxy-2-isopropyl-7-[4-methoxy-3-(3-methoxypropoxy)benzyl]-8-methylnonanamid, is known as an inhibitor of the enzyme renin. It was described for the first time in U.S. Pat. No. 5,559,111. Aliskiren may be used for the treatment of hypertension.

U.S. Pat. No. 6,737,430 discloses a mutual prodrug of amlodipine and atorvastatin, pharmaceutically acceptable acid addition salts thereof, pharmaceutical compositions thereof and the use of said prodrug and its salts in the manufacture of medicaments for the treatment of various diseases.

WO 2005/056534 A1 discloses degradation products of the statin compound rosuvastatin as well as their use as a reference standard (including reference marker) for the analysis of rosuvastatin.

WO2006/008091 A2 discloses oxidative degradation products of atorvastatin calcium and a process of the preparation thereof. Furthermore, this document is directed to atorvastatin calcium, which is substantially free of oxidative degradation products and to pharmaceutical compositions containing such atorvastatin calcium.

### SUMMARY OF THE INVENTION

The new compound, which is formed by a chemical reaction between the compounds atorvastatin and aliskiren, was discovered in the development of a combined preparation comprising atorvastatin/aliskiren. It is formed in the preparation, in mixtures at increased humidity and temperature, and in solutions of atorvastatin and aliskiren.

The new compound may be in the form of a salt. Salts of compounds having salt-forming groups are especially acid addition salts, salts with bases or, where several salt-forming groups are present, can also be mixed salts or internal salts. Salts are especially the pharmaceutically acceptable or non-toxic salts of compounds of formula.

However, for isolation and purification purposes, it is also possible to use pharmaceutically unacceptable salts.

In the following, the chemical reaction between the active substances atorvastatin and aliskiren is schematically illustrated:

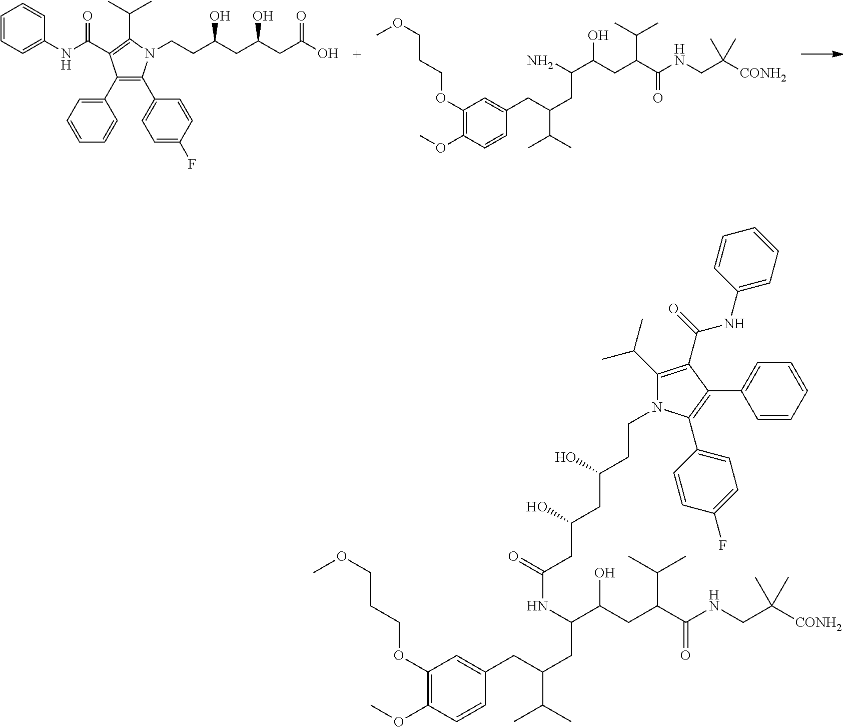

In the process for the preparation of the new compound according to the invention, the reaction between both active substances was carried out in a solution of the compounds in acetonitrile at 60° C. for 10 days. The formation of the reaction products was monitored by HPLC and HPLC-MS analyses. Using a preparative chromatography (P-HPLC), the new compound atorvastatin-aliskiren was isolated from the reaction mixture.

The compound has been spectroscopically characterised by using Mass Spectrometry (MS) and Nuclear Magnetic Resonance (NMR) spectroscopy and infrared (IR) spectroscopy.

The present invention provides a new compound in which atorvastatin and aliskiren are covalently bound, which can be used as a reference standard for analysis of either atorvastatin or aliskiren or a combination formulation of these two active substances. That is, the isolated compound may be used as an impurity standard for the purposes of quality control analyses and stability of the finished product.

Side products, byproducts and adjunct reagents (herein sometimes called impurities) are identified by physical methods and the impurities are associated with a peak position in a chromatogram and/or a spot on a TLC plate. Thereafter, the impurity can be identified by its position in the chromatogram, which is usually measured as the time period between the injection of the sample on a column and the elution of the particular component as detected by a detector, which is the retention time. This time period varies based upon the condition of the instrumentation and other factors. In order to diminish the effect of such variations, the relative retention time (RRT) is used to identify impurities. The RRT of an impurity is its retention time divided by the retention time of a reference marker.

Since the pure substance(s), i.e. atorvastatin and/or aliskiren, is/are present in such high proportion in the mixture that it tends to saturate a column, which may lead to irreproducible retention times. Thus, an alternative compound may be selected that is added to, or is present in, the mixture in an amount significant enough to be detected and sufficiently low as not to saturate the column. This compound serves as the reference marker.

The person skilled in the art understand that a compound in a relatively pure state can be used as a reference standard to quantify the amount of the compound in an unknown mixture. A reference marker is similar to a reference standard but it is used for qualitative analysis. When the compound is used as an external standard, a solution of a known concentration of the compound is analyzed by the same technique as the unknown mixture. As a result, the amount of the compound in the mixture can be determined by comparing the magnitudes of the detector response for the respective compounds.

The reference standard compound also can be used to quantify the amount of another compound in the mixture if the “response factor”, which compensates for differences in the sensitivity of the detector to the two compounds, has been predetermined. For this purpose, the reference standard compound may be added directly to the mixture as an internal standard. The reference standard compound can even be used as an internal standard when the unknown mixture contains some of the reference standard compound by using a technique called “standard addition”, wherein at least two samples are prepared by adding known and differing amounts of the internal standard. The proportion of detector response due to the reference standard compound that is originally in the mixture can be determined by extrapolation of a plot of detector response versus the amount of the reference standard compound that was added to each of the samples to zero. In this connection reference is made to Strobel, H. A.; Heineman, W. R., Chemical Instrumentation: A Systematic Approach, 3rd dd. (Wiley & Sons: New York 1989); and Snyder, L. R.; Kirkland, J. J. Introduction to Modern Liquid Chromatography, 2nd ed. (John Wiley & Sons: New York 1979).

Beside the above described use as an impurity standard, the present invention is also directed to the use of the above compound in which atorvastatin and aliskiren are covalently bound, as a potential prodrug for atorvastatin and/or aliskiren, or a pharmaceutically acceptable salt thereof, as a medicine.

Specifically, the invention is directed to the above compound in which atorvastatin and aliskiren are covalently bound or a pharmaceutically acceptable salt thereof for the treatment of the conditions described herein by providing aliskiren and atorvastatin in vivo by cleavage of the covalent bound between atorvastatin and aliskiren.

The invention is also directed to the use of the above described compound in which atorvastatin and aliskiren are covalently bound or a pharmaceutically acceptable salt thereof, for the treatment of the conditions described herein.

The invention is also directed to the use of compound in which atorvastatin and aliskiren are covalently bound, or a pharmaceutically acceptable salt thereof, in the manufacture of a medicament for the treatment of the conditions described herein.

The invention is specifically directed to the compound in which atorvastatin and aliskiren are covalently bound or a pharmaceutically acceptable salt thereof for the treatment of a disease selected from the group consisting of vascular disorder, inflammatory disease, allergic disease, neurodegenerative disease, malignant disease, viral disease, abnormal bone state, amyloid-[beta] precursor protein processing disorder such as Alzheimer's disease or Down's Syndrome, dyslipidemia, hyperlipidemia, hypercholesterolemia, atherosclerosis, arteriosclerosis, coronary artery disease, angina pectoris and/or for the management of cardiac risk.

The invention is specifically directed to the use of compound in which atorvastatin and aliskiren are covalently bound or a pharmaceutically acceptable salt thereof for the treatment of combined hypertension and hyperlipidaemia.

The invention is also directed to the use of compound in which atorvastatin and aliskiren are covalently bound or a pharmaceutically acceptable salt thereof in the manufacture of a medicament combined with atorvastatin, or a pharmaceutically acceptable salt thereof, and/or aliskiren, or a pharmaceutically acceptable salt thereof, for the treatment of the conditions described herein.

The invention is also directed to a process for the preparation of a compound in which atorvastatin and aliskiren are covalently bound, which comprises the reaction of atorvastatin and aliskiren in a solution of acetonitrile at an elevated temperature, i.e. above room temperature (>23° C.) for a suitable time period. Preferably, the reaction between both active substances is carried out in a solution of the compounds in acetonitrile at 60° C. for 10 days.

The invention is also directed to a pharmaceutical composition comprising the above compound in which atorvastatin and aliskiren are covalently bound, or a pharmaceutically acceptable salt thereof, and a pharmaceutically acceptable excipient, diluent or carrier.

The invention is also directed to a method for analyzing a sample of atorvastatin and/or aliskiren comprising the steps of a) performing chromatography on the sample to obtain data; and b) comparing the data with the chromatography data of above compound in which atorvastatin and aliskiren are covalently bound, or a salt thereof.

Preferably, this method may comprises the following steps: (a) preparing a solution of atorvastatin and/or aliskiren containing above compound in which atorvastatin and aliskiren are covalently bound, or a salt thereof;(b) subjecting the solution to a high pressure liquid chromatography to obtain a chromatogram; and (c) comparing a peak obtained in the chromatogram to a peak resulting from above compound in which atorvastatin and aliskiren are covalently bound, or a salt thereof.

Alternatively, this method may comprises the following steps: (a) preparing a solution of atorvastatin and/or aliskiren containing above compound in which atorvastatin and aliskiren are covalently bound, or a salt thereof; (b) subjecting the solution to thin layer chromatography to obtain a chromatogram; and (c) comparing a band or spot obtained in the chromatogram to a peak or band resulting from the above compound in which atorvastatin and aliskiren are covalently bound, or a salt thereof.

Furthermore, the invention is directed to a method for determining the retention time of a chromatography column for atorvastatin and/or aliskiren, comprising the steps of carrying out chromatography with above compound in which atorvastatin and aliskiren are covalently bound, or a salt thereof, as a standard.

The novel compounds according to the present invention prepared and isolated by the methods as described above were structurally characterized by Mass Spectrometry (MS) and Nuclear Magnetic Resonance (NMR) spectroscopy in order to determine the chemical structure of the said novel compounds. The methods of characterization and their results are presented in the examples described below. Using P-HPLC, a new impurity (atorvastatin-aliskiren) was isolated and its structure was determined with the help of MS and NMR measurements. For the characterization of the new compound mass spectrometry (MS), Nuclear Magnetic Resonance (NMR) spectrometry and infrared (IR) analyses were carried out.

### BRIEF DESCRIPTION OF THE FIGURES

FIG. 1: Chromatogram of the preparative separation of the original sample. Fraction 1 is labeled.

FIG. 2: Chromatogram of the preparative separation of fraction 1 shown in FIG. 1. Fraction 1/1 is labeled.

FIG. 3: UV spectrum of the finished product.

FIG. 4: Analytical chromatograms of the original sample and of the finished product.

FIG. 5: IR spectrum of the sample AT-AL-170507.

FIG. 6: ¹H NMR spectrum of the sample AT-AL-170507.

### EXAMPLES

### Example 1

500 mg of atorvastatin calcium and 500 mg of aliskiren were dissolved in 100 ml of acetonitrile ad heated at 60° C. for 10 days. The reaction mixture was diluted with 100 ml of water and injected on a preparative HPLC system, consisted of a column, ID 50 mm, length 200 mm, filled with Luna C18 prep 10 μm stationary phase, a UV detector at 300 nm and two pumps, providing 140 ml/min of total flow. Water was used as mobile phase A and acetonitrile as mobile phase B. Initial mobile phase composition was 80% water 20% acetonitrile. This composition was kept constant for 20 seconds after the injection, then it was changed to 30% water 70% acetonitrile in next 10 seconds, and then to 25% water 75% acetonitrile in next 210 seconds. A fraction was collected from 260 seconds to 280 seconds. The collected fraction was then evaporated and the residue dried in vacuum. 50 mg of atorvastatin-aliskiren compound, with chromatographic purity of 96% area, was obtained.

### Example 2

### 1. P-HPLC (Preparative HPLC) Isolation

From the reaction mixture (prepared as described in Example 1) atorvastatin-aliskiren was isolated with the help of P-HPLC. The reaction mixture was diluted with water (1:1) and directly applied to the P-HPLC.

### Chromatographic Conditions of the Basic Preparative Separation:

| System: | Varian | |
|---|---|---|
| Column: | Luna 10 μm prep C18(2) (200 × 50) mm | |
| Mobile phase A: | water | |
| Mobile phase B: | acetonitrile | |
| Flow rate: | 140 ml/min | |
| | Time (min) | % B |
| Gradient: | 0 | 20 |
| | 0:20 | 20 |
| | 0:30 | 70 |
| | 4:00 | 75 |
| | 4:20 | 90 |
| | 5:40 | 90 |
| | 5:50 | 20 |

| | |
|---|---|
| Sample application: | 200 ml |
| Detector: | 300 nm |

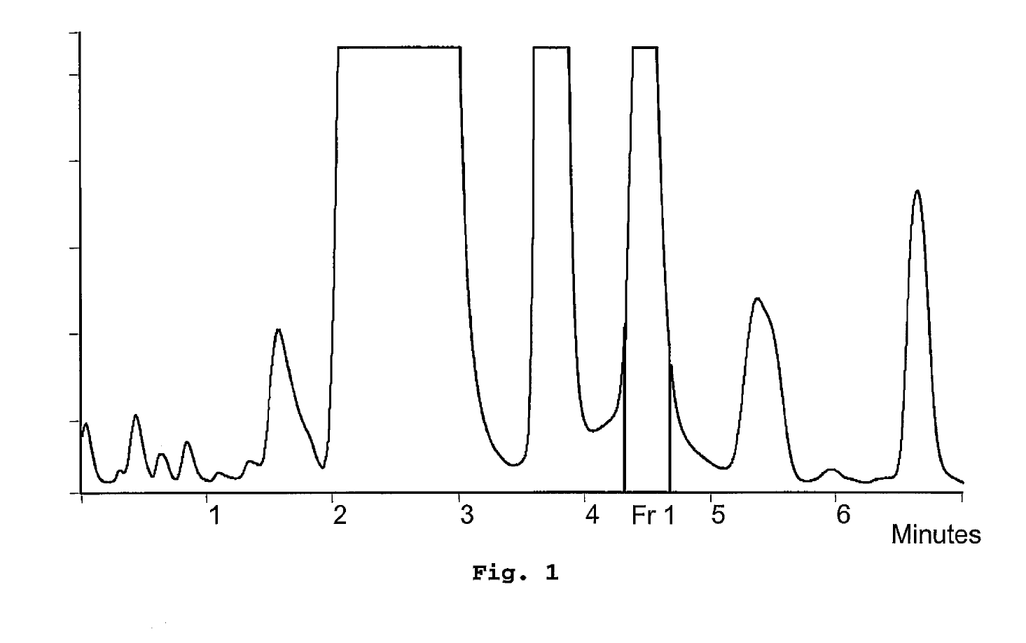

Collected was one fraction which was partially evaporated and chromatographed again on the identical system as in the original sample; also this time, one fraction was collected. FIG. 1 shows a chromatogram of the preparative separation of the original sample. The labeled peak is the fraction 1.

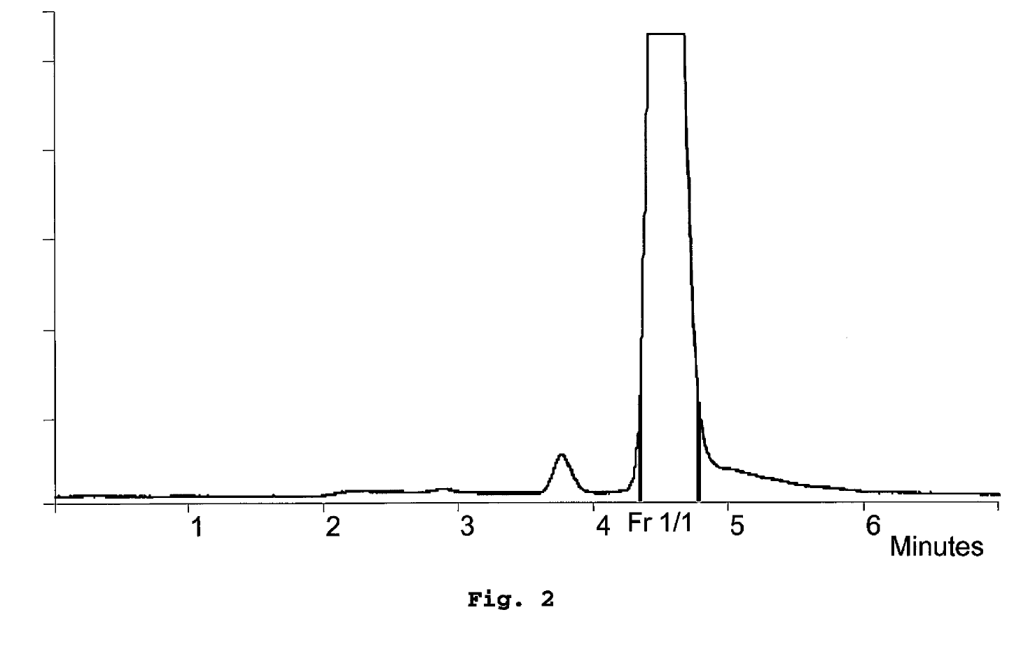

Fraction 1/1 was evaporated to dryness and dried in vacuum. FIG. 2 shows a chromatogram of the preparative separation of the above fraction 1. Fraction 1/1 is labeled. Designation of the sample product: AT-AL-170507

### Analysis of the Finished Product

### 1.1. HPLC

### Chromatographic Conditions

| System: | HP 1100 Q | |
|---|---|---|
| Column: | Chromolith Performance RP18e (150 × 4.6) | |
| | mm | |
| T_{of the column}: | 30° C. | |
| Mobile phase A: | 10 mM ammonium acetate pH 7/25% ACN/ | |
| | 5% THF | |
| Mobile phase B: | 10 mM ammonium acetate pH 7/70% ACN/ | |
| | 5% THF | |
| Flow rate: | 7 ml/min | |
| | Time (min) | % B |
| Gradient: | 0 | 26 |
| | 2 | 26 |
| | 4.7 | 100 |

| | |
|---|---|
| Detector: | 248 nm |

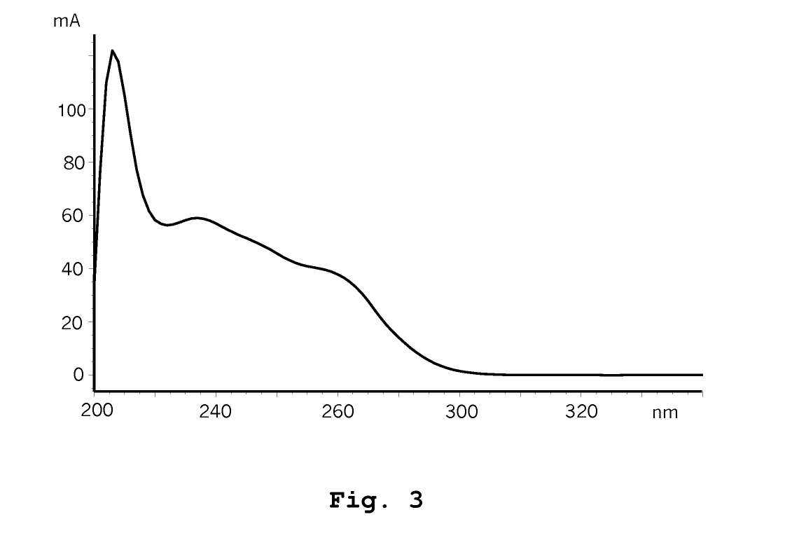

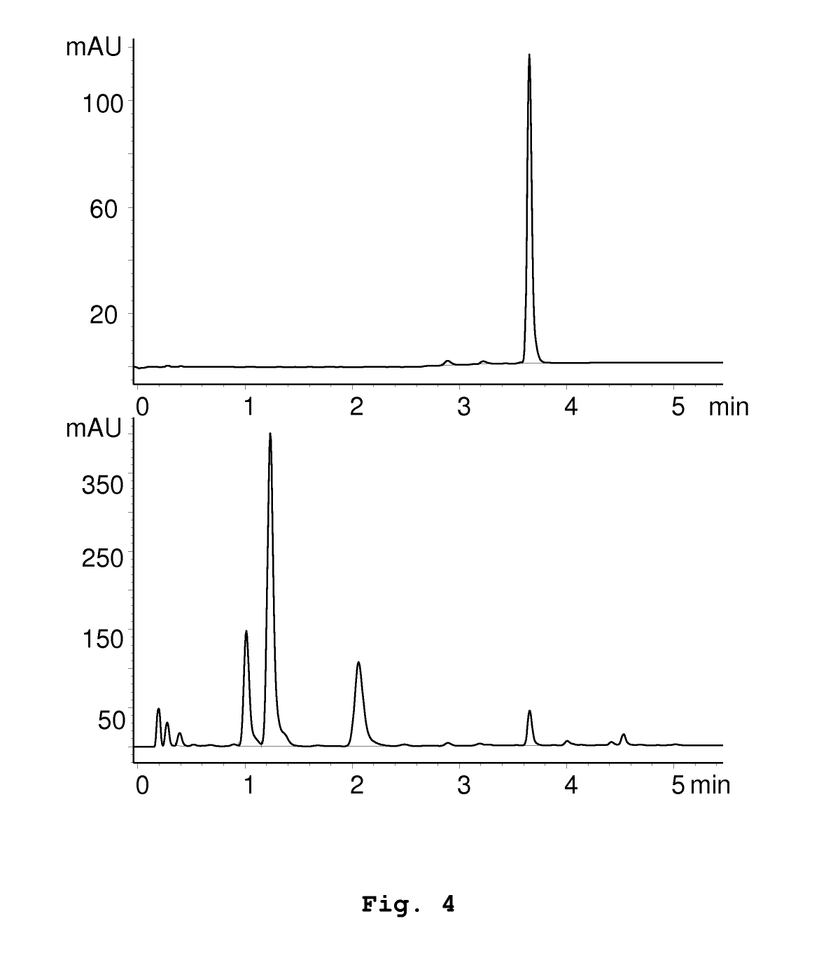

FIG. 3 shows an UV spectrum of the finished product. FIG. 4 shows analytical chromatograms of the original sample and of the finished product.

### 1.2. IR Spectrometric Analysis

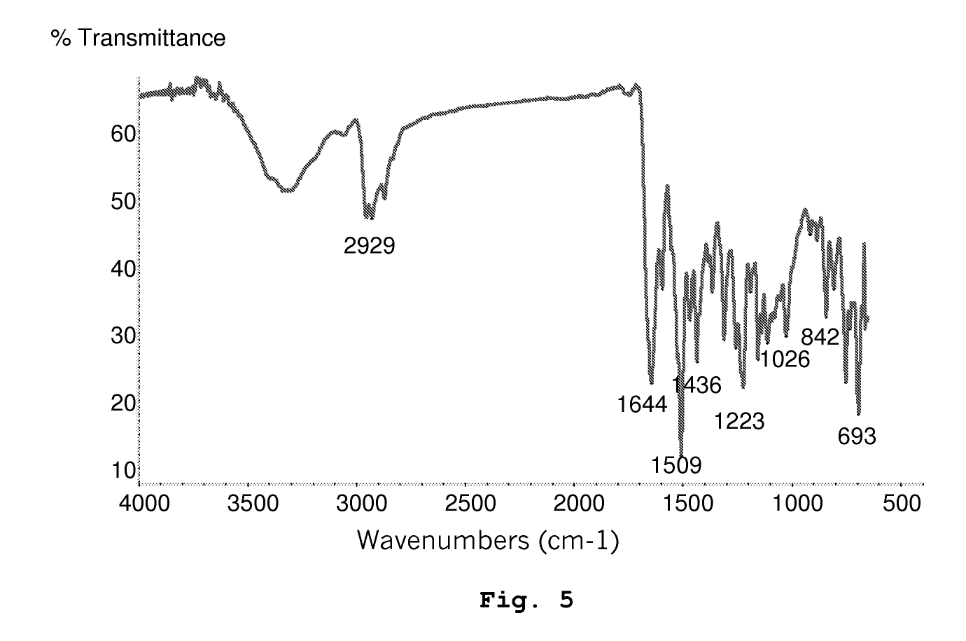

IR spectrum of the finished product was recorded. IR spectrum was recorded on the FTIR instrument Nexus, Themo Nicolet, using ATR technique with ZnSe crystal. FIG. 5 shows an IR spectrum of the sample compound AT-AL-170507.

### 1.3. Mass Spectrometry Analysis

The finished product was analyzed using a HPLC-MS system and a MS/MS spectrum was recorded.

### HPLC-MS Conditions

| HPLC system: | AT1100QTOF | |
|---|---|---|
| Column: | Zorbax Eclipse XDB C8 3.5 μm | |
| | (150 × 4.6) mm | |
| T_{of the column}: | 30° C. | |
| Mobile phase A: | 20 mM ammonium acetate pH 4/ | |
| | 5% ACN | |
| Mobile phase B: | 20 mM ammonium acetate pH 4/ | |
| | 75% ACN | |
| Flow rate: | 7 ml/min | |
| | Time (min) | % B |
| Gradient: | 0 | 20 |
| | 10 | 50 |
| | 22 | 50 |
| | 30 | 66 |
| | 35 | 100 |
| | 40 | 100 |

| | |
|---|---|
| V_{of injection}: | 10 μl |
| Split of the flow rate in MS: | 1:7 |
| MS instrument: | Micromass Q-TOF Ultima Global |
| Ionisation: | ESI, positive |
| T_{source}: | 120° C. |
| T_{des}: | 350° C. |
| Cone gas: | 200 L/h |
| Des. gas | 650 L/h |
| MCP: | 1900 V |
| TOF geometry: | V |

### MS/MS Conditions

| | |
|---|---|
| MS instrument: | Micromass Q-TOF Ultima Global |
| Ionisation: | ESI, positive |
| T_{source}: | 80° C. |
| T_{des}: | 120° C. |
| Cone gas: | 0 L/h |
| Des. gas | 200 L/h |
| MCP: | 1900 V |
| TOF geometry: | V |
| Colliding gas: | Ar (10 psi) |
| Colliding energy: | 22 eV |
| Application of the sample: | direct infusion, 5 μl/min |
| Sample: | prepared in 10 mM ammonium |
| | acetate/50% ACN, |
| | concentration 10⁻² mg/ml |

Using HPLC-MS conditions described in chapter 1.3. MASS SPECTROMETRY ANALYSIS chromatographic peak for AT-AL-170507 is detected at retention time 33.9 min. In mass spectrum protonated compound atorvastatin-aliskiren (M+H)⁺=1092.7 m/z is observed. Visible are also adducts of the compound with sodium (M+Na)⁺=1114.7 m/z, with potassium (M+K)⁺=1130.7 m/z and with ammonium and acetonitrile (M+MeCN+NH₄)⁺=1150.8 m/z.

In MS/MS spectrum of the ion (M+H)⁺=1092.7 m/z following major characteristic fragment ions are detected 1074.6 m/z, 999.6 m/z, 982.6 m/z, 883.5 m/z.

### 1.4. NMR Spectrometry Analysis

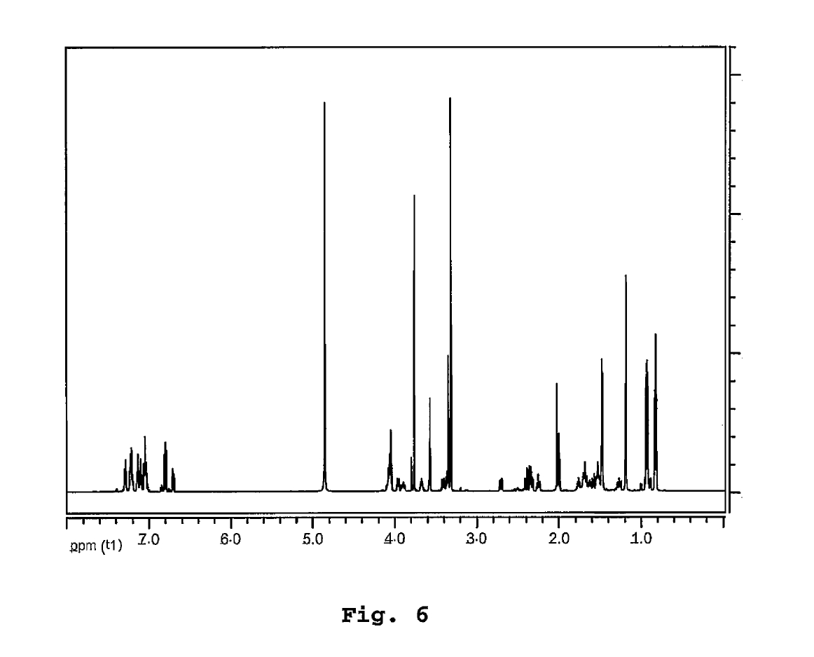

The sample was dissolved in CD₃OD. NMR spectra were recorded on a 600 MHz instrument Varian Unity Inova600. As a reference, residual signal CD₃OD was used (3.31 ppm in ¹H NMR spectrum or 49.15 ppm in ¹³C NMR spectrum). Recorded were ¹H and ¹³C spectra and two-dimensional H—H, H—C correlation spectra. FIG. 6 shows the ¹H NMR spectrum of the compound AT-AL-170507 corresponding to atorvastatin-aliskiren.

On the basis of obtained results, the following structure is proposed:

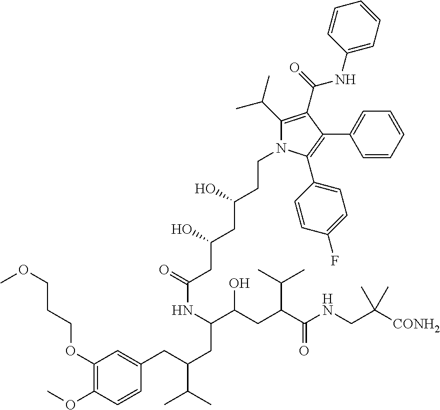

Structure of the inventive compound atorvastatin-aliskiren.

1. A compound in which atorvastatin and aliskiren are covalently bound, having the formula 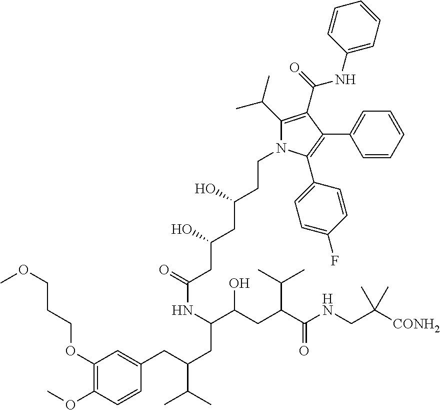
or a salt thereof.

2. A compound according to claim 1 or a pharmaceutically acceptable salt thereof for use as a medicine.

3. The compound according to claim 2, or a pharmaceutically acceptable salt thereof, for the treatment of one or more diseases selected from the group consisting of hypertension, vascular disorder, inflammatory disease, allergic disease, neurodegenerative disease, malignant disease, viral disease, abnormal bone state, amyloid-[beta] precursor protein processing disorder such as Alzheimer's disease or Down's Syndrome, dyslipidemia, hyperlipidemia, hypercholesterolemia, atherosclerosis, arteriosclerosis, coronary artery disease, angina pectoris and/or for the management of cardiac risk.

4. A compound according to claim 3, or a pharmaceutically acceptable salt thereof, for the treatment of combined hypertension and hyperlipidaemia.

5. The use of a compound according to claim 1, or a salt thereof as an impurity standard.

6. A process for the preparation of a compound in which atorvastatin and aliskiren are covalently bound, which process comprises
the reaction of atorvastatin and aliskiren in a solution of acetonitrile at an elevated temperature.

7. A pharmaceutical composition comprising the compound according to claim 1, or a pharmaceutically acceptable salt thereof, and a pharmaceutically acceptable excipient, diluent or carrier.

8. A method for analyzing a sample of atorvastatin and/or aliskiren comprising the steps of:
a) performing chromatography on the sample to obtain data; and
b) comparing the data with the chromatography data of the compound of claim 1.

9. The method of claim 8, wherein the method comprises the following steps:
(a) preparing a solution of atorvastatin and/or aliskiren containing the compound of claim 1;
(b) subjecting the solution to a high pressure liquid chromatography to obtain a chromatogram; and
(c) comparing a peak obtained in the chromatogram to a peak resulting from the compound of claim 1.

10. The method of claim 8, wherein the method comprises the following steps:
(a) preparing a solution of atorvastatin and/or aliskiren containing the compound of claim 1;
(b) subjecting the solution to thin layer chromatography to obtain a chromatogram; and
(c) comparing a band or spot obtained in the chromatogram to a peak or band resulting from the compound of claim 1.

11. A method for determining the retention time on a chromatographic system for atorvastatin and/or aliskiren,
comprising the steps of carrying out chromatography with the compound of claim 1 as a standard.
